(19) United States
(12) Patent Application Publication
(10) Pub. No.: US 2025/0215020 A1
(43) Pub. Date: Jul. 3, 2025

(54) COMPOUNDS AS ELASTASE INHIBITOR AND METHOD OF PREPARING THE SAME

(71) Applicant: SHANGHAI ARK BIOPHARMACEUTICAL CO. LTD., Shanghai (CN)

(72) Inventors: Weikai GU, Shanghai (CN); Mixia WANG, Shanghai (CN); Feifei GE, Shanghai (CN); Qiwu ZHAO, Shanghai (CN); Haiqing YUAN, Shanghai (CN)

(21) Appl. No.: 19/002,149

(22) Filed: Dec. 26, 2024

(30) Foreign Application Priority Data
Dec. 29, 2023 (CN) 202311866336.1

(51) Int. Cl. C07F 5/02

(52) U.S. Cl. CPC C07F 5/025

(57) ABSTRACT

The present disclosure relates to a process for preparing an elastase inhibitor compound. The process is: friendly to the environment, simple in operation, highly efficient, mild in reaction conditions, easy to control in operation, safe and reliable, low-cost and good in economic benefits. Meanwhile, the process provided in present disclosure offers a high reaction yield and a high purity for the obtained product, thus being suitable for industrial production.

### TECHNICAL FIELD

The present disclosure belongs to the field of medicine, and particularly relates to a preparation process of an elastase inhibitor compound.

### BACKGROUND

Human neutrophil elastase (HNE), also known as human leukocyte elastase (HLE), is a 32 kDa serine protease and a member of the neutrophil serine protease (NSPs) family. Currently, HNE is considered to be a multifunctional enzyme with various proteolytic activities. It can participate in pathogen killing, inflammation regulation, and maintenance of tissue homeostasis, thereby becoming the important protective barrier when the body is threatened. However, excessive NHE is highly destructive to the human body because the over-released HNE will cause direct tissue damage by hydrolyzing its specific substrates, and lead to symptoms of acute infections, acute hemolysis and blood loss, tissue damage or necrosis, acute poisoning and malignant tumors by participating inflammatory reactions in various organs. As a result, the liver specifically synthesizes and secretes a natural inhibitor (α−1 antitrypsin) to maintain the balance of the elastase activity in the human body.

Elastin is one of the main components of the extracellular matrix protein, and other components include fibronectin, laminin, proteoglycan, type III and type IV collagen. The functions of the elastase include acting against bacterial invasions by degrading bacterial structural proteins. In short, the human elastase can degrade damaged tissues or invading bacteria and thus plays an indispensable role in maintaining the body's homeostasis.

The imbalance of elastase content is a basic characteristic of serious diseases, especially those related to the cardiopulmonary system, such as chronic obstructive pulmonary disease (COPD), bronchiectasis (BE), cystic fibrosis (CF), acute lung injury (ALI), acute respiratory distress syndrome (ARDS), pulmonary artery hypertension (PAH), and idiopathic pulmonary fibrosis (IPF). When inflammation occurs, unlike under normal physiological conditions, activated neutrophils release excessive HNE, upregulate cell adhesion factors to promote the adhesion of neutrophils to endothelial cells, and release pro-inflammatory molecules to induce cytokine expression. These substances can in turn activate neutrophils to release more HNE. Among them, chronic obstructive pulmonary disease is a disease with high incidence. Currently, there are approximately 100 million patients with chronic obstructive pulmonary disease in China, and this number is still increasing due to factors such as population aging and air pollution. There is no effective treatment for this disease so far, and the currently used bronchodilators (LABA/LAMA) can only relieve symptoms without stopping the progression of the disease. These patients will still continuously suffer from pulmonary infections, and after each episode of inflammation, the lung function impairment will become more severe.

At present, there are extensive researches on using elastase inhibitors to treat this type of inflammatory disease abroad, and the first marketed drug, sivelestat sodium hydrate, is the first successfully developed drug that treats such diseases by elastase inhibition. In addition, several inhibitors have entered or are in clinical trials to test their therapeutic effects on diseases such as chronic obstructive pulmonary disease, cystic fibrosis, or α−1 antitrypsin deficiency.

However, the existing compounds have a short in vivo exposure time and are rapidly eliminated, which may weaken their efficacy. Therefore, it is necessary to develop a class of elastase inhibitors with extended in vivo exposure time and improved exposure amount.

### SUMMARY

### Problems to be Solved by Present Invention

To address the above-mentioned problems in the prior art, provided in present disclosure is a process for preparing an elastase inhibitor compound that is easy to operate, economical, suitable for industrial production, high in reaction yield, and has an improved in vivo exposure and an extended half-life.

### Solutions to the Problem

Provided in present disclosure is a process for preparing a compound of formula (I), including:

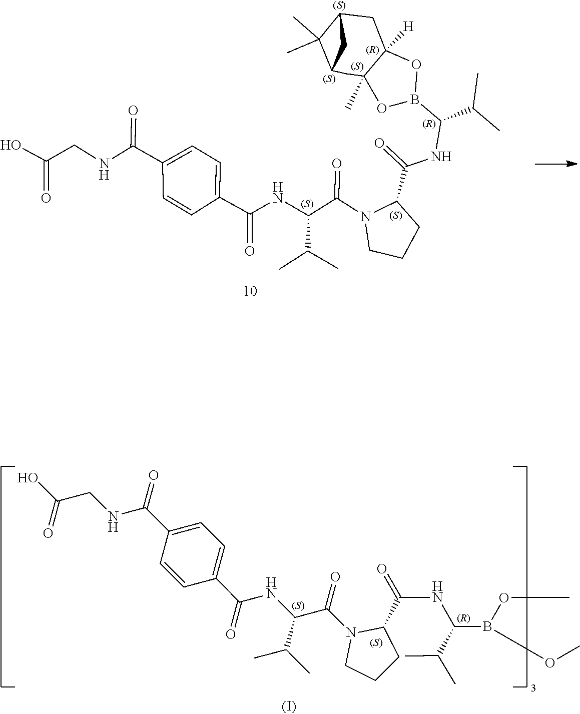

Step S7: reacting Compound 10 in the presence of a borylation reagent, an acidification reagent and a reaction solvent to obtain the compound of formula (I).

Preferably, wherein the borylation reagent is selected from the group consisting of isobutyl boronic acid, phenylboronic acid and 4-trifluorophenylboronic acid.

Preferably, the acidification reagent is selected from the group consisting of trifluoroacetic acid, hydrochloric acid, sulfuric acid and phosphoric acid.

Preferably, the reaction solvent is selected from the group consisting of acetonitrile, acetone, dichloromethane, 1,4-dioxane, methyl tert-butyl ether, alkane solvents (n-heptane, n-hexane, methylcyclohexane, petroleum ether), water, and combinations thereof.

Further, for the process as described above, step S7 further includes a post-treatment including crystallization.

Preferably, the crystallization solvent is selected from the group consisting of 1,4-dioxane, tetrahydrofuran, 2-methyltetrahydrofuran, methyl tert-butyl ether, ethylene glycol dimethyl ether, dichloromethane, dimethyl sulfoxide, acetone, acetonitrile, water and combinations thereof.

Preferably, the solvent used for the crystallization is selected from the group consisting of a mixture of methyl tert-butyl ether, acetonitrile and water, a mixture of acetonitrile and water and a mixture of acetone and water.

Further, for the process as described above, the process further includes:

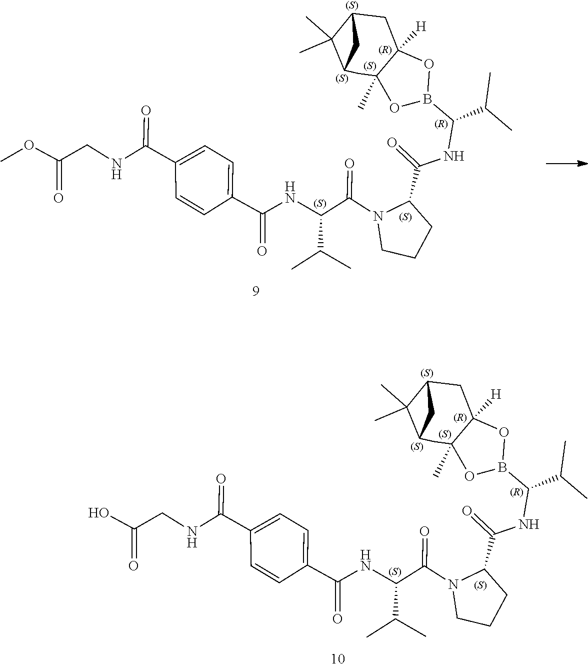

Step S6: reacting Compound 9 in the presence of a base to obtain Compound 10.

Further, for the process as described above, the base is selected from the group consisting of sodium hydroxide, potassium hydroxide, lithium hydroxide and combinations thereof.

Preferably, the base is lithium hydroxide.

Further, for the process as described above, step S6 further includes a post-treatment step including an extraction.

Preferably, the extraction includes the addition of water and an extractant into the reaction solution for extracting after the reaction is completed.

Preferably, the extractant is selected from the group consisting of ethyl acetate, cyclopentyl methyl ether, isopropyl acetate, methyl tert-butyl ether, methyl acetate, propyl acetate and combinations thereof; preferably, the extractant is methyl tert-butyl ether.

Further, the post-treatment step of step S6 includes crystallization.

Preferably, the crystallization solvent is selected from the group consisting of 1,4-dioxane, n-heptane, tetrahydrofuran, ethyl acetate, isopropyl acetate, 2-methyltetrahydrofuran, methyl tert-butyl ether, ethylene glycol dimethyl ether, dichloromethane, dimethyl sulfoxide, acetonitrile and combinations thereof; more preferably, the crystallization solvent is a mixture of n-heptane and tetrahydrofuran.

Further, for the process as described above, the process further includes:

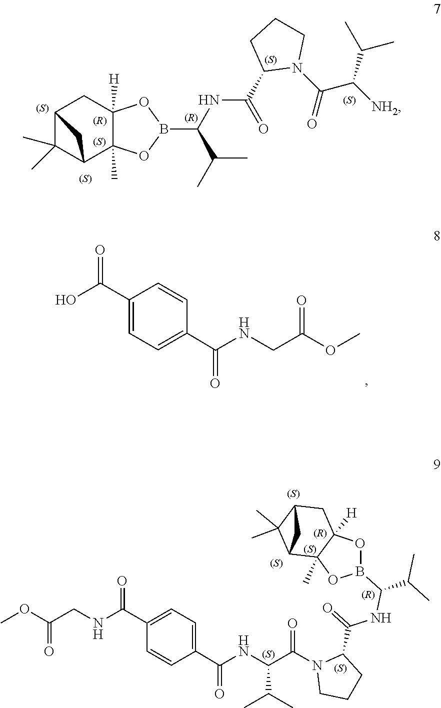

Step S5: reacting Compound 7 or the salt thereof with compound 8 in the presence of a first condensation reagent to obtain compound 9.

Further, for the process as described above, the first condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof.

Preferably, the first condensation reagent is T₃P or T₄P.

Preferably, the first condensation reagent is T₄P.

Further, for the process as described above, the process further includes:

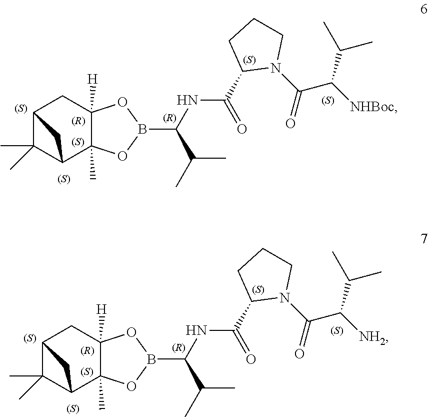

Step S4: reacting Compound 6 in the presence of an acid to obtain a salt of compound 7.

Further, for the process as described above, the acid is selected from the group consisting of hydrochloric acid, trifluoroacetic acid (TFA), sulfuric acid, p-toluenesulfonic acid, formic acid and combinations thereof; preferably, the acid is hydrochloric acid.

Further, for the process as described above, the process further includes:

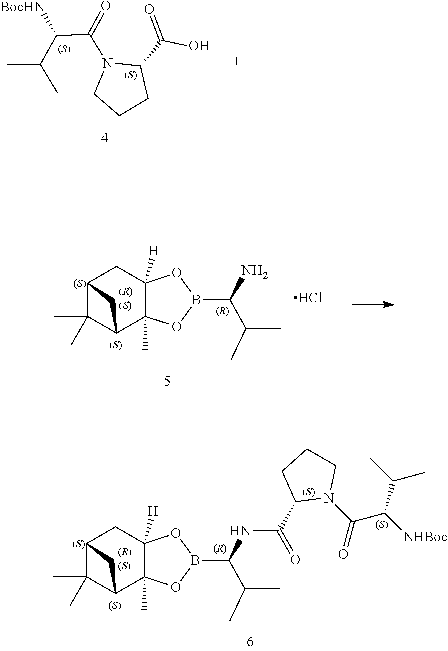

Step S3: reacting Compound 4 with compound 5 in the presence of a second condensation reagent to obtain compound 6.

Further, for the process as described above, the second condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof. Preferably, the second condensation reagent is T₃P or T₄P. Preferably, the second condensation reagent is T₄P.

### EFFECTS OF THE DISCLOSURE

(1) The creative discovery in present disclosure is that compound 10 reacts to obtain the trimer compound of formula (I) in the presence of isobutylboronic acid and trifluoroacetic acid.

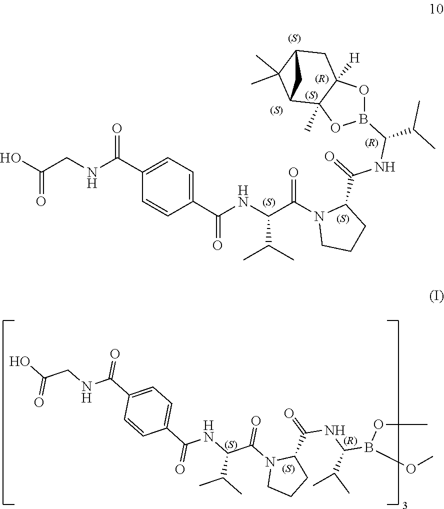

(2) The process provided in the present disclosure is friendly to the environment, simple in operation, highly efficient, mild in reaction conditions, easy to control in operation, safe and reliable, low-cost and good in economic benefits. Meanwhile, the process provided in present disclosure offers a high reaction yield and a high purity for the obtained product, thus being suitable for industrial production.
(3) In the preparation process provided in the present disclosure, hydrochloric acid used as the deprotecting agent in step S4 offers a high reaction conversion rate and product purity, and the obtained solid compound 7 in hydrochloride salt form enables to simplify the subsequent separation and purification, facilitating the scale-up production.
(4) In the preparation process provided in the present disclosure, the T₃P or T₄P is combined with DIPEA/THF to form the condensation reaction system in steps S5 and S3, which suppresses the racemization of the reaction, and improves the reaction conversion rate and yield. At the same time, due to the high purity of the product, the workup is simple, and the tetrahydrofuran solution of the reaction product can be directly used for the subsequent reaction without the step of purification using column chromatography, facilitating the scale-up production.
(5) In the preparation process provided in the present disclosure, the stability of reaction temperature, the equivalence of alkalinity and reaction time is examined in step S6. It reduces the risk of product racemization, and improves the reaction purity. Besides, the developed crystallization process under the conditions of tetrahydrofuran and n-heptane in step S6 effectively increases the purity of the obtained product and facilitates the scale-up production and the storage of intermediates.
(6) In the preparation process provided in the present disclosure, the deprotection reaction in step S7 is carried out in the presence of isobutyl boronic acid/trifluoroacetic acid, which improves the solubility of the raw materials for reaction and prevents the agglomeration of the raw materials during the reaction, and refrains from the concentration and water removal process during the workup. Meanwhile, the crystallization process using ACN/MTBE allows to improve the purity of the product and reaction yield, facilitating scale-up production.

### BRIEF DESCRIPTION OF THE DRAWINGS

FIG. 1 shows the high-resolution mass spectrum of the compound shown in Formula (I).

### DETAILED DESCRIPTION

To make the technical solutions and beneficial effects of present disclosure more obvious and easier to understand, the following description is explained in detail by enumerating specific embodiments. The drawings are not necessarily drawn to scale, and local features can be enlarged or reduced to show the details of local features more clearly. Unless otherwise defined, the technical and scientific terms used in this text have the same meanings as those in the technical field to which present disclosure belongs.

Provided in present disclosure is a process for preparing a compound of formula (I), including:

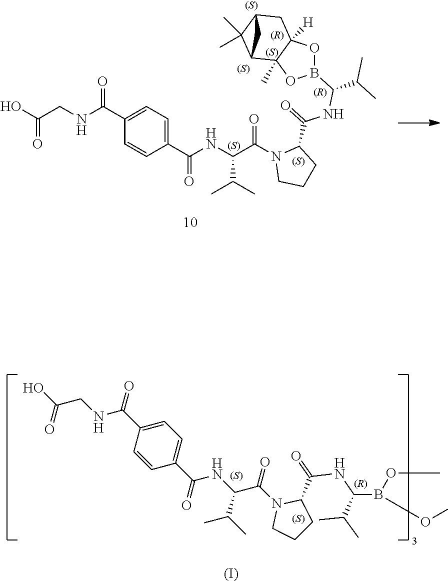

Step S7: reacting Compound 10 in the presence of a borylation reagent, an acidification reagent and a reaction solvent to obtain the compound of formula (I).

In some embodiments, the borylation reagent is selected from the group consisting of isobutyl boronic acid, phenylboronic acid and 4-trifluorophenylboronic acid.

In some embodiments, the borylation reagent is isobutyl boronic acid.

In some embodiments, the acidification reagent is selected from the group consisting of trifluoroacetic acid, hydrochloric acid, sulfuric acid and phosphoric acid.

In some embodiments, the acidification reagent is trifluoroacetic acid.

In some embodiments, the reaction solvent is selected from the group consisting of acetonitrile, acetone, dichloromethane, 1,4-dioxane, methyl tert-butyl ether, alkane solvents (n-heptane, n-hexane, methylcyclohexane, petroleum ether), water, and combinations thereof.

In some embodiments, the reaction temperature of step S7 is 10-50° C.

In some embodiments, the reaction temperature of step S7 is 25-35° C.

In some embodiments, the reaction temperature of step S7 is 30° C.

In some embodiments, the reaction temperature of step S7 is 35-45° C.

In some embodiments, the reaction temperature of step S7 is 40° C.

In some embodiments, the molar ratio of compound 10 to trifluoroacetic acid to isobutyl boronic acid in step S7 is 1:(0.1-3):(0.5-9).

In some embodiments, the molar ratio of compound 10 to trifluoroacetic acid to isobutyl boronic acid in step S7 is 1:(0.5-1.5):(1.5-4.5).

In some embodiments, the molar ratio of compound 10 to trifluoroacetic acid to isobutyl boronic acid in step S7 is 1:1:3.

In some embodiments, the workup of step S7 includes crystallization.

In some embodiments, the crystallization step includes the addition of a crystallization solvent to the reaction solution after the reaction is completed.

In some embodiments, the crystallization solvent is selected from the group consisting of 1,4-dioxane, tetrahydrofuran, 2-methyltetrahydrofuran, methyl tert-butyl ether, ethylene glycol dimethyl ether, dichloromethane, dimethyl sulfoxide, acetone, acetonitrile, water and combinations thereof.

In some embodiments, the crystallization solvent is selected from the group consisting of a mixture of methyl tert-butyl ether, acetonitrile and water, a mixture of acetonitrile and water and a mixture of acetone and water.

In some embodiments, the crystallization solvent is a mixture of acetonitrile and water.

In some embodiments, the crystallization step is carried out at 0-20° C.

In some embodiments, the crystallization step is carried out at 0-15° C.

In some embodiments, the crystallization step is carried out at 0-10° C.

In some embodiments, the crystallization step is carried out at 5° C.

In some embodiments, the process further includes the following steps:

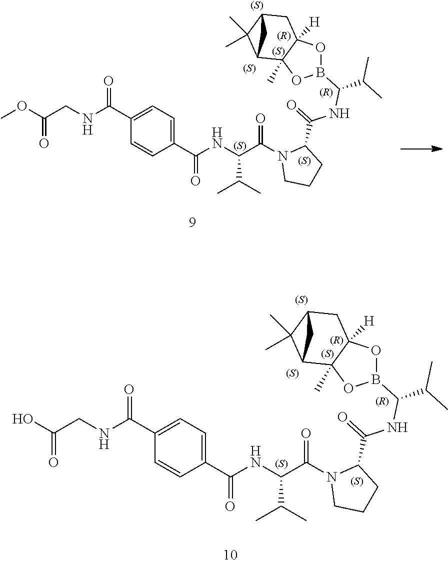

Step S6: reacting Compound 9 in the presence of a base to obtain Compound 10.

In some embodiments, the base is selected from the group consisting of sodium hydroxide, potassium hydroxide, lithium hydroxide and combinations thereof.

In some embodiments, the base is lithium hydroxide.

In some embodiments, the molar ratio of compound 9 to the base in step S6 is 1:(0.5-3).

In some embodiments, the molar ratio of compound 9 to the base in step S6 is 1:(1-2).

In some embodiments, the molar ratio of compound 9 to the base in step S6 is 1:1.35.

In some embodiments, the reaction temperature of step S6 is 0-10° C.

In some embodiments, the reaction temperature of step S6 is 5° C.

In some embodiments, the workup of step S6 includes an extraction step.

In some embodiments, the extraction step includes the addition of water and an extractant into the reaction solution for extracting after the reaction is completed.

In some embodiments, the extractant is selected from the group consisting of ethyl acetate, cyclopentyl methyl ether, isopropyl acetate, methyl tert-butyl ether, methyl acetate, propyl acetate and combinations thereof.

In some embodiments, the extractant is methyl tert-butyl ether.

In some embodiments, the extraction step is carried out at 0-10° C.

In some embodiments, the extraction step is carried out at 5° C.

In some embodiments, the workup of step S6 includes a crystallization step.

In some embodiments, the crystallization step includes adding a crystallization solvent to the reaction solution after the reaction is completed.

In some embodiments, the crystallization solvent is selected from the group consisting of 1,4-dioxane, n-heptane, tetrahydrofuran, ethyl acetate, isopropyl acetate, 2-methyltetrahydrofuran, methyl tert-butyl ether, ethylene glycol dimethyl ether, dichloromethane, dimethyl sulfoxide, acetonitrile and combinations thereof.

In some embodiments, the crystallization solvent is the mixture of n-heptane and tetrahydrofuran.

In some embodiments, the crystallization is carried out at 0-20° C.

In some embodiments, the crystallization is carried out at 5-15° C.

In some embodiments, the crystallization is carried out at 10° C.

In some embodiments, the process further includes the following steps:

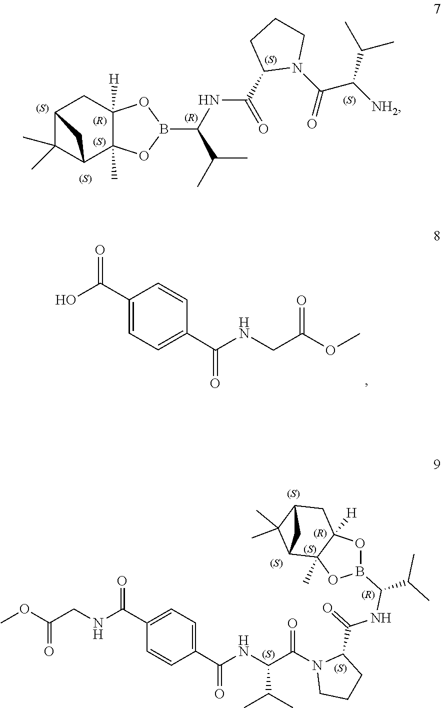

Step S5: Compound 7 or the salt thereof reacts with compound 8 in the presence of a first condensation reagent to obtain compound 9.

In some embodiments, the first condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof.

In some embodiments, the first condensation reagent is T₃P or T₄P.

In some embodiments, the first condensation reagent is T₄P.

In some embodiments, the condensation reaction in step S5 is carried out in the presence of a base and a solvent.

In some embodiments, the base is an organic base.

In some embodiments, the base is selected from the group consisting of TEA, DIPEA, N-methylmorpholine, and pyridine.

In some embodiments, the base is DIPEA.

In some embodiments, the solvent is selected from tetrahydrofuran, ethyl acetate, acetonitrile, acetone, dichloromethane, N,N-dimethylformamide, N,N-dimethylacetamide, and pyridine.

In some embodiments, the solvent is tetrahydrofuran.

In some embodiments, the condensation reaction in step S5 is carried out in the presence of DIPEA and tetrahydrofuran.

In some embodiments, step S5 is carried out in the reaction system of T₃P/DIPEA/THF.

In some embodiments, the molar ratio of compound 7 or its salt to compound 8 to T₃P to DIPEA in step S5 is 1:(0.5-1.5):(1-3):(3-7).

In some embodiments, the molar ratio of compound 7 or its salt to compound 8 to T₃P to DIPEA in step S5 is 1:(1-1.2):(1.5-2.5):(4-6).

In some embodiments, the molar ratio of compound 7 or its salt to compound 8 to T₃P to DIPEA in step S5 is 1:1.05:2:5.

In some embodiments, step S5 is carried out in the reaction system of T₄P/DIPEA/THF.

In some embodiments, the molar ratio of compound 7 or its salt to compound 8 to T₄P to DIPEA in step S5 is 1:(0.5-1.5):(1-3):(3-7).

In some embodiments, the molar ratio of compound 7 or its salt to compound 8 to T₄P to DIPEA in step S5 is 1:(1-1.2):(1.5-2.5):(4-6).

In some embodiments, the molar ratio of compound 7 or its salt to compound 8 to T₄P to DIPEA in step S5 is 1:1.05:2:5.

In some embodiments, the reaction temperature of step S5 is −60˜−10° C.

In some embodiments, the reaction temperature of step S5 is −50˜−20° C.

In some embodiments, the reaction temperature of step S5 is −40˜−30° C.

In some embodiments, the process further includes the following steps:

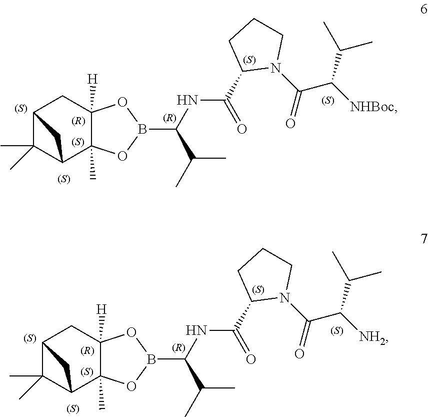

Step S4: reacting Compound 6 in the presence of an acid to obtain a salt of compound 7.

In some embodiments, the acid is selected from the group consisting of hydrochloric acid, trifluoroacetic acid (TFA), sulfuric acid, p-toluenesulfonic acid, phosphoric acid, formic acid and combinations thereof.

In some embodiments, the acid is hydrochloric acid.

In some embodiments, the molar ratio of compound 6 to the acid in step S4 is 1:(3-9).

In some embodiments, the molar ratio of compound 6 to the acid in step S4 is 1:(5-7).

In some embodiments, the molar ratio of compound 6 to the acid in step S4 is 1:6.36.

In some embodiments, the reaction temperature of step S4 is 20-60° C.

In some embodiments, the reaction temperature of step S4 is 30-40° C.

In some embodiments, the reaction temperature of step S4 is 35° C.

In some embodiments, the process further includes the following steps:

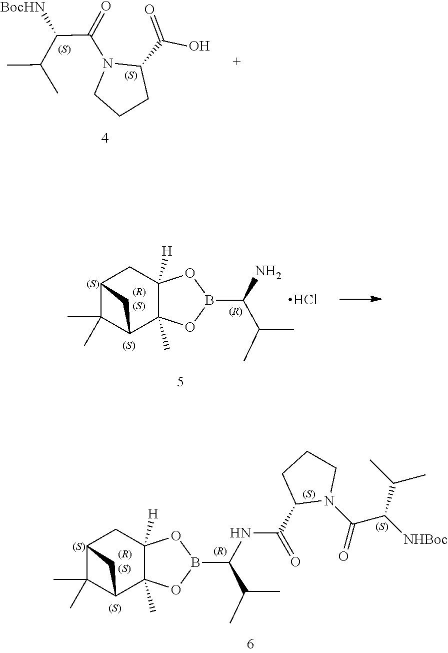

Step S3: reacting Compound 4 with compound 5 in the presence of a second condensation reagent to obtain compound 6.

In some embodiments, the second condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof.

In some embodiments, the second condensing reagent is T₃P or T₄P.

In some embodiments, the second condensing reagent is T₄P.

In some embodiments, the condensation reaction in step S3 is carried out in the presence of a base and a solvent.

In some embodiments, the base is an organic base.

In some embodiments, the base is selected from the group consisting of TEA, DIPEA, N-methylmorpholine and pyridine.

In some embodiments, the base is DIPEA.

In some embodiments, the solvent is selected from the group consisting of tetrahydrofuran, ethyl acetate, acetonitrile, acetone, dichloromethane, N,N-dimethylformamide, N,N-dimethylacetamide, and pyridine.

In some embodiments, the solvent is tetrahydrofuran.

In some embodiments, the condensation reaction in step S3 is carried out in the presence of DIPEA and tetrahydrofuran.

In some embodiments, step S3 is carried out in the reaction system of T₃P/DIPEA/THF.

In some embodiments, the molar ratio of compound 4 and compound 5 to T₃P to DIPEA in step S3 is 1:(0.5-1.5):(1-3):(3-7).

In some embodiments, the molar ratio of compound 4 and compound 5 to T₃P to DIPEA in step S3 is 1:(1-1.2):(1.5-2.5):(4-6).

In some embodiments, the molar ratio of compound 4 and compound 5 to T₃P to DIPEA in step S3 is 1:1.05:2:5.

In some embodiments, step S3 is carried out in the reaction system of T₄P/DIPEA/THF.

In some embodiments, the molar ratio of compound 4 and compound 5 to T₄P to DIPEA in step S3 is 1:(0.5-1.5):(1-3):(3-7).

In some embodiments, the molar ratio of compound 4 and compound 5 to T₄P to DIPEA in step S3 is 1:(1-1.2):(1.5-2.5):(4-6).

In some embodiments, the molar ratio of compound 4 and compound 5 to T₄P to DIPEA in step S3 is 1:1.05:2:5.

In some embodiments, the reaction temperature of step S3 is −80˜20° C.

In some embodiments, the reaction temperature of step S3 is selected from −70˜−60° C., −60˜−50° C., −30˜−20° C., or 0˜10° C.

In some embodiments, the reaction temperature of step S3 is −80˜−30° C.

In some embodiments, the reaction temperature of step S3 is −70˜−40° C.

In some embodiments, the reaction temperature of step S3 is −60˜−50° C.

In some embodiments, the process further includes the following steps:

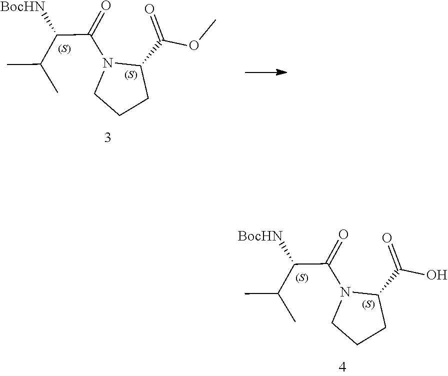

Step S2: reacting Compound 3 in the presence of a base to form compound 4.

In some embodiments, the base is selected from the group consisting of sodium hydroxide, potassium hydroxide, lithium hydroxide and combinations thereof.

In some embodiments, the base is lithium hydroxide.

In some embodiments, the molar ratio of compound 3 to the base in step S2 is 1:(0.5-5).

In some embodiments, the molar ratio of compound 3 to the base in step S2 is 1:(1-3).

In some embodiments, the molar ratio of compound 3 to the base in step S2 is 1:2.

In some embodiments, the reaction temperature of step S2 is 0-40° C.

In some embodiments, the reaction temperature of step S2 is 0-10° C., 30-35° C., or 20-25° C.

In some embodiments, the reaction temperature of step S2 is 0-5° C.

In some embodiments, the process further includes the following step:

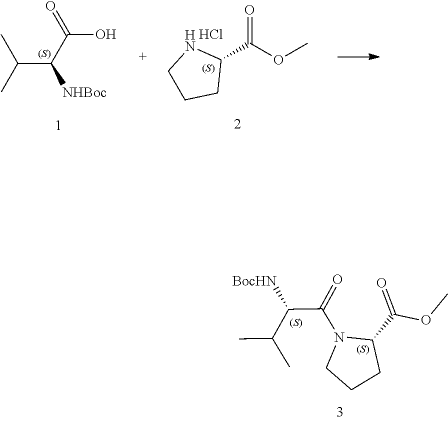

Step S1: reacting Compound 1 with compound 2 in the presence of a third condensation reagent to obtain compound 3.

In some embodiments, the third condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof.

In some embodiments, the third condensation reagent is CDI.

In some embodiments, the molar ratio of compound 1 to compound 2 to CDI to DIPEA in step S1 is 1:(0.5-1.5):(0.5-1.5):(1-3).

In some embodiments, the molar ratio of compound 1 to compound 2 to CDI to DIPEA in step S1 is 1:(0.8-1.2):(0.8-1.2):(1.5-2.5).

In some embodiments, the molar ratio of compound 1 to compound 2 to CDI to DIPEA in step S1 is 1:1.1:1.05:2.

In some embodiments, the reaction temperature of step S1 is 10-40° C.

In some embodiments, the reaction temperature of step S1 is 20-30° C. or 30-35° C.

In some embodiments, the reaction temperature of step S1 is 25° C.

The following embodiments are described for illustrating the process of present disclosure. It should be understood that these embodiments are used to explain the basic principles, main features, and advantages of present disclosure that is not limited by the scope of the following embodiments. The experimental conditions adopted in the embodiments can be further adjusted according to specific requirements, and the unstated experimental conditions are usually those in conventional experiments.

In the following examples, the ¹H NMR spectra were measured using a Bruker instrument (400 MHZ), and expressed in chemical shifts by ppm, and D20 was used as the internal standard (0.00 ppm). The ¹H NMR is represented as: s=singlet, d=doublet, t=triplet, q=quartet, m=multiplet, br=broad peak, dd=doublet of doublets, dt=doublet of triplets. The unit of the coupling constant is Hz when provided.

The mass spectra were measured using MALDI-8030 MALDI-TOF MS, and the ionization mode was negative ions.

Elemental analysis was carried out using a Vario Micro Cube fully automatic elemental analyzer and a Leeman Prodigy inductively coupled plasma emission spectrometer. C, H, and N were detected using a fully automatic elemental analyzer; B was tested using ICP-OES; O was calculated according to the results of C, H, N, and B based on the law of conservation of mass.

Purity was analyzed using HPLC, Agilent 1260 II, equipped with a UV detector.

In the following examples, unless otherwise specified, all temperatures are in Celsius. Unless otherwise specified, all starting materials and reagents are either commercially available or synthesized according to known methods. Commercially available raw materials and reagents are used directly without further purification, unless otherwise specified. Commercially available manufacturers include but are not limited to Sinopharm Group, J&K Scientific Ltd., Tokyo Chemical Industry (Shanghai) Development Co., Ltd., Shanghai Bide Pharmaceutical Technology Co., Ltd., and Shanghai Meryer Biochemical Co., Ltd., etc.

Unless otherwise specified in the examples, the solutions in the reactions refer to aqueous solutions.

Unless otherwise specified in the examples, the reaction temperature is room temperature, which is 20° C.-30° C.

The English abbreviations of the compounds are shown in Table 1 below:

TABLE 1

| | | | | | |
|---|---|---|---|---|---|
| MTBE | Methyl tert-butyl ether | DCM | Dichloromethane | TFA | Trifluoroacetic acid |
| DIPEA | N,N- | TEA | Triethylamine | THF | Tetrahydrofuran |
| | Diisopropylethylamine | | | | |
| HCl | Hydrogen chloride | EA | Ethyl acetate | LiOH | Lithium hydroxide |
| CDI | N,N′- | T₃P | Propylphosphonic | T₄P | n-Butylphosphonic |
| | Carbonyldiimidazole | | anhydride | | anhydride |
| BOP-Cl | Bis(2-oxo-3- | DCC | Dicyclohexylcarbodiimide | DPP-Cl | Diphenylphosphoryl |
| | azolidinyl)phosphinic | | | | chloride |
| | chloride | | | | |
| PyBOP | Hexafluorophosphate | DIC | N,N′- | DPPA | Diphenylphosphorylazide |
| | benzotriazol-1-yl- | | Diisopropylcarbodiimide | | |
| | oxytripyrrolidinylphosphorus | | | | |
| HCTU | 6-Chlorobenzotriazole- | EDCI | 1-Ethyl-(3- | HATU | 2-(7-Aza- |
| | 1,1,3,3- | | dimethylaminopropyl) | | benzotriazol-1-yl)- |
| | tetramethyluronium | | carbodiimide | | N,N,N′,N′- |
| | hexafluorophosphate | | hydrochloride | | tetramethyluronium |
| | | | | | hexafluorophosphate |
| HBTU | O-Benzotriazole- | TBTU | O-Benzotriazole- | BOP | Benzotriazol-1- |
| | tetramethyluronium | | N,N,N′,N′- | | yloxytris(dimethylamino) |
| | hexafluorophosphate | | tetramethyluronium | | phosphonium |
| | | | tetrafluoroborate | | hexafluorophosphate |

### Example 1 Preparation of Compound 3

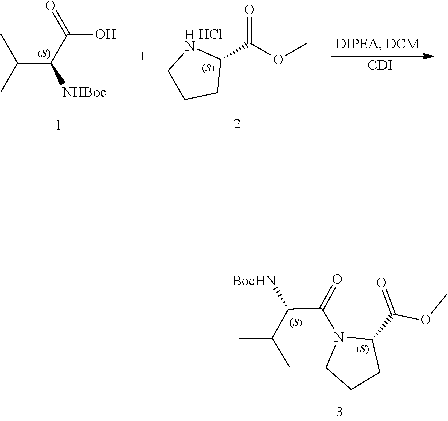

Dichloromethane (1320.0 g) was added to a dry three-necked flask and stirred under nitrogen protection following the addition of compound 2(84.0 g). Then, the temperature is controlled to 20° C., and DIPEA (2.0 eq, 119.0 g) was added dropwise to the three-necked flask under nitrogen protection. After the addition, the mixture was stirred for 1-2 h and marked as system R1. Dichloromethane (1320.0 g) was added to a dry jacketed flask, stirred under nitrogen protection and added with 100.0 g of compound 1. The temperature was controlled to 20° C., and CDI (1.05 eq, 78.4 g) was added dropwise in batches following with stirring at 25° C. for 2-4 h. Then, the solution of system RI was added to the jacketed flask with the temperature kept at 10-30° C. and stirred at 25° C. for 5-7 h. After the reaction was completed, the reaction mixture was added with water (200.0 g) under stirring at a temperature below 25° C. and placed for stratification. Then, the resulting organic phase was transferred to the reactor under stirring, was added with 1 mol/L hydrochloric acid aqueous solution (1000.0 g) under stirring at a temperature of 20° C. and placed for stratification. Then, the resulting organic phase was transferred to the reactor under stirring, added with 10% sodium bicarbonate aqueous solution (1000.0 g) at a temperature of 20° C. under stirring and placed for stratification. The resulting organic phase was transferred to the reactor under stirring, added with water (500.0 g) at a temperature of 25° C. under stirring, and placed for stratification. The resulting organic phase was concentrated under reduced pressure to 100-200 mL, obtaining a colorless to pale yellow solution. Tetrahydrofuran (5 V, 446.0 g) was added to the solution that was then concentrated under reduced pressure to 100-200 mL. Then, tetrahydrofuran (89.0 g) was added again to the solution that was stirred to obtain a colorless to pale yellow solution. The solution contains 120.0 g of compound 3 with a yield of 79.4% and a purity of ≥95% by calculation.

The characterization data of compound 3:
¹H NMR (400 MHz, DMSO-d6) δ6.88-6.37 (m, 1H), 4.32 (dd, J=8.5, 5.4 Hz, 1H), 403 (t, J=8.3 Hz, 1H), 3.77 (d, J=9.9 Hz, 1H), 3.61 (s, 4H), 2.28-2.08 (m, 1H), 2.04-1.75(m, 4H), 1.37 (s, 9H), 0.89 (dd, J=19.2, 6.7 Hz, 6H).
MS [M+H]⁺=329

### Example 2 Preparation of Compound 4

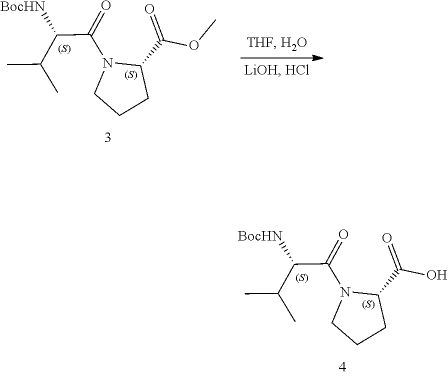

Water (131.0 g) and lithium hydroxide monohydrate (25.5 g) were added to a beaker and stirred to dissolve for later use. The jacketed flask containing tetrahydrofuran (267.0 g) was added with the solution (1.00 eq, 100.0 g) of compound 3 in tetrahydrofuran (450 mL) under stirring and nitrogen protection, to which the lithium hydroxide aqueous solution was added dropwise under at 0-5° C. and kept stirring for 2-3 h. After the reaction was completed, the reaction solution was added with 2% sodium hydroxide aqueous solution (265 g) at a temperature of 0-5° C., and then with methyl tert-butyl ether (222.0 g) under stirring. The resulting solution was placed for stratification and given the aqueous phase (W1) for the next step and the organic phase (O1) for temporary storage. The aqueous phase (W1) was added with Methyl tert-butyl ether (222.0 g) at 20° C., stirred and placed for stratification to give the aqueous phase (W2) for temporary storage. The organic phase (O1) was transferred to the extraction reactor, added with water (200 g) under stirring and placed for stratification to give the aqueous phase (W3) for temporary storage. The combined aqueous phase (W2 and W3) was transferred to the reactor under stirring, added with methyl tert-butyl ether (148.0 g) at 20° C. under stirring, and placed for stratification. The resulting aqueous phase was added to the reaction flask following with addition of dichloromethane (585.2 g) under stirring, cooled down to 0-5° C., added with 2 mol/L hydrochloric acid aqueous solution (355.0 g) dropwise to adjust the pH to 3-4 under stirring, and placed for stratification. The resulting aqueous phase was added with dichloromethane (264.0 g) under stirring, placed for stratification to give the organic phase that was then concentrated to about 100 mL under reduced pressure to obtain a colorless to pale yellow solution. This solution was added with n-heptane (680.0 g) dropwise, stirred for 2 h, cooled down to 0-10° C., stirred for 1-2 h, and filtered. The filter cake was washed with n-heptane (68.0 g) and dried to give a white to off-white solid (compound 4, 83.0 g) with a yield of 86.7% and a purity of ≥97.0%.

The characterization data of compound 4:
¹H NMR (400 MHZ, DMSO-d₆) δ12.33 (s, 1H), 6.79 (d, J=8.6 Hz, 1H), 4.24 (dd, J=8.6, 5.0 Hz, 1H), 4.01 (t, J=8.3 Hz, 1H), 3.82-3.69 (m, 1H), 3.56 (dt, J=9.7, 6.6 Hz, 1H), 2.16 (ddt, J=12.1, 8.7, 7.1 Hz, 1H), 1.92 (d, J=6.2 Hz, 3H), 1.82 (dq, J=12.2, 6.4, 5.8 Hz, 1H), 1.37 (s, 10H), 0.89 (dd, J=20.8, 6.7 Hz, 6H).
MS [M+Na]⁺=337

### Example 3-1 Preparation of Compound 6

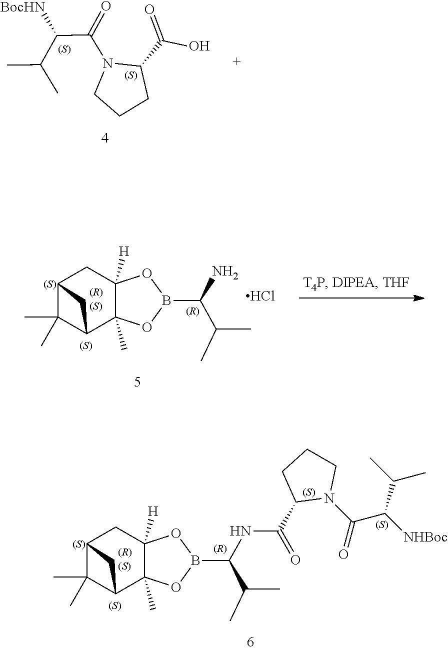

Under nitrogen protection, compound 4 (1.00 eq, 2.10 g), compound 5 (1.00 eq, 1.91 g) and THF (21 g) were successively added, and cooled down to −50 to −60° C. following with addition of DIPEA (4.3 g) dropwise, stirred for 10-30 min at this temperature, and added with 50% T₄P ethyl acetate solution (9.5 g) dropwise. After the addition, the solution was allowed to heat up naturally to a temperature ranging from −20° C. to 0° C. for the reaction. The reaction solution was transferred to the reactor after the reaction was completed. Potassium carbonate (2.1 g) was added to purified water (18 g) and dissolved under stirring for later use. The prepared 10% potassium carbonate aqueous solution was added to the reactor dropwise at 5° C. Then, reaction solution was added with MTBE (15 g) stirred and placed for stratification to give the organic phase 3-A for temporary storage. MTBE (7.5 g) was again added to the resulting aqueous phase which was stirred and placed for stratification to give the organic phase 3-B for temporary storage. The organic phases (3-A and 3-B) were combined to give the organic phase 3-C. Concentrated hydrochloric acid (1.6 g) was added to purified water (18 g) and stirred well for later use. The organic phase 3-C was added with the prepared hydrochloric acid aqueous solution at 0° C., stirred and placed for stratification to give the organic phase 3-D. The organic phase 3-D was added with purified water (10 g) at 0° C. for extraction twice. The resulting organic phase was concentrated until there was no obvious distillate left. THF (10 g) was added and dissolved in this solution that was concentrated until there was no obvious distillate left. Then, repeat this operation. THF (8.0 g) was added to the resulting solution and stirred to give THF solution of compound 6 that can be directly used in the next step. The THE solution of compound 6 was tested by HPLC and calculated to obtain 3.14 g of compound 6 at a purity of 98% and a yield of 90%.

The characterization data of compound 6:
¹H NMR (400 MHZ, DMSO-d₆) δ8.86 (t, J=3.3 Hz, 1H), 6.84 (d, J=8.5 Hz, 1H), 4.49 (dd, J=8.6, 4.3 Hz, 1H), 4.09-3.95 (m, 2H), 3.75-3.55 (m, 2H), 2.25-1.58 (m, 13H), 1.37 (s, 9H), 1.21 (d, J=6.9 Hz, 6H), 0.92-0.80 (m, 15H).
MS [M+H]⁺=548

### Examples 3-2 to 3-5 Preparation of Compound 6

Examples 3-2, 3-3, 3-4 and 3-5 were carried out with reference to Example 3-1. Their experimental conditions and results are shown in Table 2 below.

| | | Reaction |
|---|---|---|
| Example | | Conversion |
| No. | Reaction Reagents and Dosages | Rate (%) |
| 3-2 | Compound 4 (20 g, 1.0 equivalent), | 90.8 |
| | Compound 5: 1.0 equivalent; | |
| | DIPEA: 5 equivalents; | |
| | T₃P: 2.8 equivalents | |
| | Acetonitrile: 400 mL | |
| | Reaction temperature: −20° C. to 0° C. | |
| | Reaction time: 2 hours | |
| 3-3 | Compound 4 (20 g, 1.0 equivalent), | 96.2 |
| | Compound 5: 1.0 equivalent; | |
| | DIPEA: 5 equivalents; | |
| | T₃P: 2.0 equivalents | |
| | THF: 400 mL | |
| | Reaction temperature: −40° C. to −30° C. | |
| | Reaction time: 2 hours | |
| 3-4 | Compound 4 (10 g, 1.0 equivalent), | 96.2 |
| | Compound 5: 1.0 equivalent; | |
| | DIPEA: 5 equivalents; | |
| | T₃P: 2.0 equivalents | |
| | Ethyl acetate (EA): 200 mL | |
| | Reaction temperature: −40° C. to −30° C. | |
| | Reaction time: 2 hours | |
| 3-5 | Compound 4 (20 g, 1.0 equivalent), | 96 |
| | Compound 5: 1.0 equivalent; | |
| | DIPEA: 5 equivalents; | |
| | T₄P: 2.0 equivalents | |
| | THF: 400 mL | |
| | Reaction temperature: −40° C. to −30° C. | |
| | Reaction time: 2 hours | |

### Example 4 Preparation of Compound 7

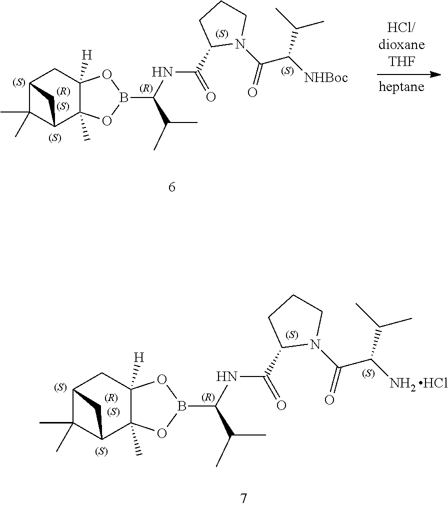

Under nitrogen protection, 24.0 g of the tetrahydrofuran solution of compound 6 (content: 25%) was heated to 30-40° C., stirred to dissolve, and added with HCl in 1,4-dioxane solution (4 mol/L, 18 g) dropwise, and stirred for 12-16 h at 35° C. After the reaction was completed, the reaction solution was concentrated under reduced pressure until there was no obvious distillate left. The concentrated residue was added with tetrahydrofuran (20 g), stirred to dissolve, concentrated under reduced pressure until there was no obvious distillate left. Repeat this operation twice. The concentrated residue was added with tetrahydrofuran (18 g), stirred to dissolve, aged for 10-12 h at 50° C., added with n-heptane slowly, cooled down to 5-15° C., and filtered. The filter cake was washed with heptane, and dried to give a white solid (5.1 g) having a purity of 99.3% and a yield of 88%.

The characterization data of compound 7:
¹H NMR (400 MHZ, DMSO-d₆) δ8.80 (t, J=3.9 Hz, 1H), 8.16 (d, J=5.2 Hz, 3H), 4.53 (dd, J=8.3, 5.4 Hz, 1H), 4.10 (dd, J=8.7, 2.2 Hz, 1H), 3.96 (t, J=5.4 Hz, 1H), 3.74 (dt, J=9.9, 6.4 Hz, 1H), 3.57-3.52 (m, 1H), 2.41 (dd, J=5.7, 3.5 Hz, 1H), 2.25-2.10 (m, 3H), 2.06-1.95 (m, 2H), 1.89-1.71 (m, 5H), 1.65 (dt, J=13.9, 2.8 Hz, 1H), 1.35 (d, J=10.1 Hz, 1H), 1.22 (d, J=2.3 Hz, 6H), 1.01 (d, J=6.9 Hz, 3H), 0.95 (d, J=6.8 Hz, 3H), 0.89 (dd, J=13.1, 6.8 Hz, 6H), 0.81 (s, 3H).
MS [M+H]⁺=448

### Example 5 Preparation of Compound 9

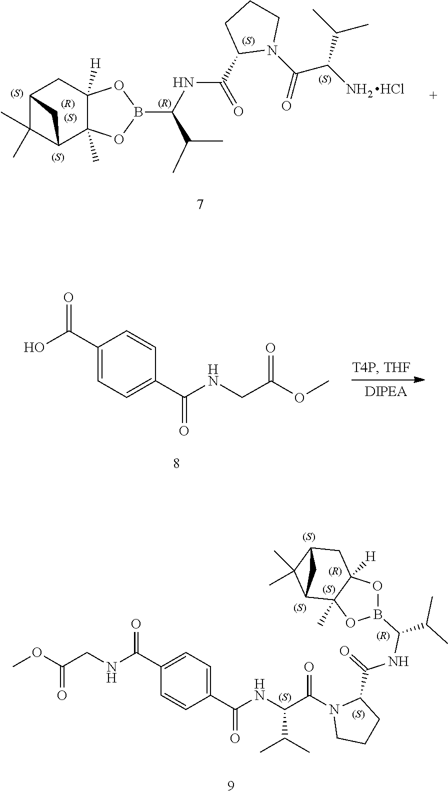

Under nitrogen protection, compound 7 (5 g), compound 8 (2.55 g) and tetrahydrofuran (45 g) were successively added, cooled down to −30 to −40° C., added with DIPEA (6.7 g) dropwise, stirred for 20-40 min, added with 50% T₄P ethyl acetate solution (14.9 g) dropwise and raised to −10 to 10° C. for the reaction. After the reaction was completed, 10% potassium carbonate aqueous solution (50 g) was added to the reaction solution dropwise. Then, methyl tert-butyl ether (27.4 g) was added and stirred. The reaction solution was placed for stratification to give the aqueous phase and the organic phase 5-A that is temporarily stored. Methyl tert-butyl ether (13.7 g) was added to the aqueous phase that was stirred, placed for stratification to give the aqueous phase and the organic phase 5-B for temporary storage. The organic phase 5-C obtained from combining organic phases 5-A and 5-B was added with IM hydrochloric acid aqueous solution (50 g), stirred, placed for stratification to give the aqueous phase and the organic phase 5-D that is temporarily stored. The organic phase 5-D was added with purified water (25 g) for extraction to give the bottom aqueous phase and the organic phase that was temporarily stored. Repeat this operation and combine the organic phases of the two extractions to give the organic phase 5-E. The organic phase 5-E was concentrated until there was no obvious distillate left. After the addition of tetrahydrofuran (20 g), the concentrated residue was concentrated under reduced pressure until there was no obvious distillate left in twice. Tetrahydrofuran (20 g) was added to the concentrated residue to give the THE solution of compound 9 that can be directly used in the next step. The THE solution of compound 9 was tested using HPLC and calculated to obtain 6.18 g of compound 9 at a purity of 96.8% and a yield of 90%.

The characterization data of compound 9:
¹H NMR (400 MHZ, DMSO-d₆) δ9.09 (t, J=5.9 Hz, 1H), 8.87 (t, J=3.5 Hz, 1H), 8.69 (d, J=8.1 Hz, 1H), 8.06-7.89 (m, 4H), 4.50 (d, J=4.0 Hz, 1H), 4.05 (dd, J=12.1, 6.1 Hz, 3H), 3.89 (dt, J=9.8, 7.1 Hz, 1H), 3.66 (d, J=8.9 Hz, 5H), 3.42 (t, J=6.4 Hz, 1H), 2.34 (dd, J=5.6, 2.9 Hz, 1H), 2.21-1.96 (m, 5H), 1.86-1.73 (m, 5H), 1.57-1.49 (m, 1H), 1.39 (d, J=10.2 Hz, 1H), 1.25-1.20 (m, 6H), 0.98 (dd, J=10.6, 6.7 Hz, 6H), 0.93-0.86 (m, 6H), 0.81 (d, J=3.2 Hz, 3H).
MS [M+H]⁺=667

### Example 6-1 Preparation of Compound 10

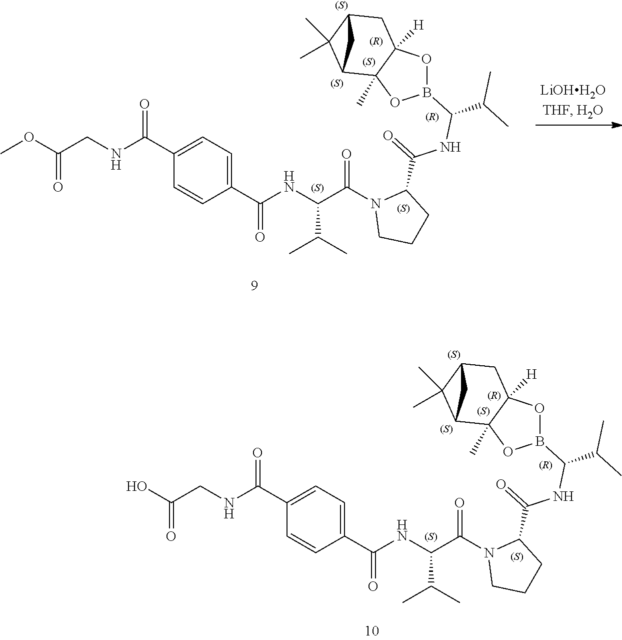

Lithium hydroxide monohydrate (0.42 g) was weighed, added and dissolved to 9.0 g of purified water (9.0 g) for later use. Under nitrogen protection, the lithium hydroxide aqueous solution was dropwise added to the reactor that contains compound 9 (5.0 g) in tetrahydrofuran, at −10° C. After that, the solution was heated to 5° C. and stirred for 10 h. After the reaction was completed, added purified water (9.0 g) to the reaction solution at 5° C., and then added MTBE (16.6 g) for extraction twice. The aqueous phase and the organic phase were separated, and the organic phase in two extractions were combined to give the organic phase 6-A which was temporarily stored. After the addition of isopropyl acetate (16.6 g) for extraction twice at 5° C., the aqueous phase was slowly added with 1M hydrochloric acid solution (11.42 g) for adjusting the pH to 2-3 and placed for separation. The organic phase obtained in this two-time extraction are collected and combined to give the organic phase 6-B. After adding purified water (15.0 g) to the combination of the organic phase 6-A and 6-B for extraction, the collected organic phase 6-C was concentrated to 5-10 mL under reduced pressure. Tetrahydrofuran (19.7 g) was added and dissolved to the concentrated residues which were then concentrated, and this operation was repeated twice. Then, after supplementary addition and dissolution of tetrahydrofuran (15.0 g) to the concentrated residues, the reactor was added with n-heptane (100.0 g), and the organic solvent (THF solution, the final weight ratio of the crystallization solvent THF to n-heptane is 1:5) dropwise. After that, the solution was stirred for 1 h and filtered, and the filter cake was washed with heptane (10.0 g), and dried to give a white solid (4 g) having a purity of 98.7% and a yield of 82%.

The characterization data of compound 10:
¹H NMR (400 MHZ, DMSO-d₆) δ12.66 (s, 1H), 8.97 (t, J=5.9 Hz, 1H), 8.87 (t, J=3.5 Hz, 1H), 8.69 (d, J=8.1 Hz, 1H), 8.02-7.90 (m, 4H), 4.57-4.44 (m, 2H), 4.07 (dd, J=8.7, 2.2 Hz, 1H), 3.95 (d, J=5.8 Hz, 2H), 3.91-3.85 (m, 1H), 3.69 (ddd, J=9.5, 7.2, 5.3 Hz, 1H), 2.34 (dd, J=5.5, 2.9 Hz, 1H), 2.25-2.07 (m, 3H), 2.03-1.89 (m, 3H), 1.86-1.72 (m, 4H), 1.65 (dt, J=13.8, 2.8 Hz, 1H), 1.39 (d, J=10.0 Hz, 1H), 1.22 (d, J=1.1 Hz, 6H), 1.11 (s, 5H), 0.98 (dd, J=10.2, 6.7 Hz, 6H), 0.90 (dd, J=13.8, 6.8 Hz, 6H), 0.81 (s, 3H).
MS [M+H]⁺=653

### Examples 6-2, 6-3 and 6-4 Preparation of Compound 10

Examples 6-2, 6-3 and 6-4 were carried out with reference to Example 6-1. Their experimental conditions and results are shown in Table 3 below.

| Example No. | Reaction Reagents and Dosages | Results |
|---|---|---|
| 6-2 | Compound 9 (3.0 g, 1.0 equivalent) | 2.7 g, off-white solid, yield: |
| | LiOH•H₂O: 2.0 equivalents | 92%, purity: 97.42% |
| | THF: 30 mL | (The solid was unstable and |
| | H₂O: 10 mL | melted into an oily substance |
| | Temperature: −10° C. to 0° C. | during filtration. An oily |
| | Crystallization solvent (weight ratio of | bubble was obtained after |
| | THF:MTBE = 1:8) | vacuum drying.) |
| 6-3 | Compound 9 (6.4 kg, 1.0 equivalent) | 6.1 kg, off-white solid, yield: |
| | LiOH.H₂O: 2.0 equivalents | 82.6%, purity: 96.2% |
| | THF: 64 L | (The solid was unstable and |
| | H₂O: 21.1 L | melted into an oily substance |
| | Temperature: −10° C. to 0° C. | during filtration. An oily |
| | Crystallization solvent (weight ratio of | bubble was obtained after |
| | ethyl acetate (EA):MTBE = 1:8) | vacuum drying.) |
| 6-4 | Compound 9 (82 g, 1.0 equivalent) | 72.2 kg, off-white solid, yield: |
| | LiOH•H₂O: 1.35 equivalents | 80.8%, purity: 98.3% |
| | THF: 246 mL | (The solid was stable) |
| | H₂O: 148 mL | |
| | Crystallization solvent (weight ratio of | |
| | THF:n-heptane = 1:5) | |
| | Temperature: −10° C. to 0° C. | |

### Example 7-1 Preparation of Compound of Formula (I)

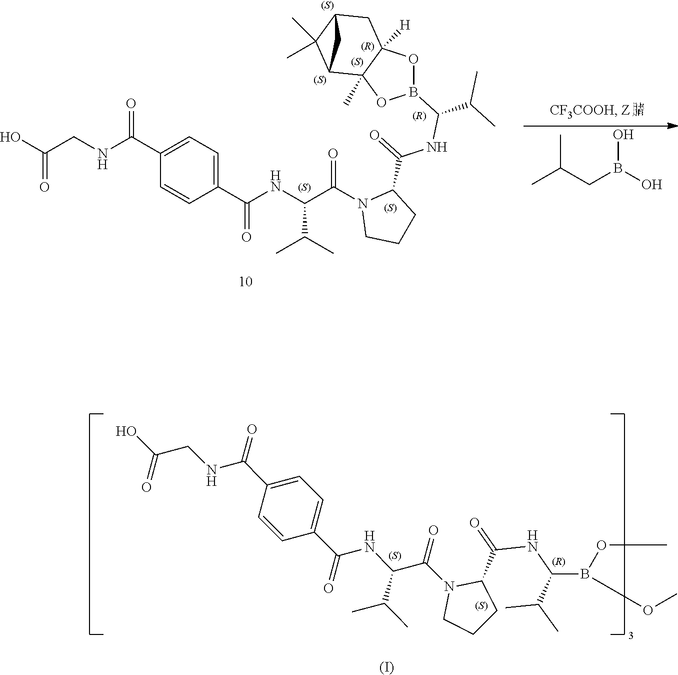

Under the protection of nitrogen, acetonitrile (12.0 g), TFA (0.87 g), isobutyl boronic acid (2.35 g) and compound 10 (5.0 g) were successively added into the reactor. After three times of nitrogen replacement, the reaction solution was stirred at 40° C. for 18-24 h. After the reaction is completed, the reaction solution was added with water (2.0 g) under stirring and then n-heptane for extraction twice (15.0 g of n-heptane was added each time). The bottom organic phase from each extraction was collected, combined and placed in a reactor that was then added with D301 basic resin (2.5 g) and stirred for 1-2 h. After removal of the resin by filtration at 30° C. and collection of the organic phase, the filtered D301 resin was washed by a mixed solution (7.5 mL) of acetonitrile and water (volume ratio of acetonitrile: water=9:1). The combination of the organic phase and the washing liquid was filtered by a precision filter and the filtrate was collected and added dropwise to the antisolvent MTBE (300 g). After that, this solution was stirred for 0.5-1 h, cooled down to 5° C., stirred for 0.5-1 h, and filtrated. The filter cake was washed with MTBE (10 g) to give a white solid (3 g) having a purity of 97.3% and a yield of 75%.

### Examples 7-2 to 7-14 Preparation of Compound of Formula (I)

Examples 7-2 to 7-14 were carried out with reference to Example 7-1. Their experimental conditions and results are shown in Table 4 below.

| | | | Conversion Rate |
|---|---|---|---|
| Example | Difference from Example 7-1 | Reaction Reagents and Conditions | (%) |
| 7-2 | Solvent for condensation | Compound 10: 5.0 g, 1.0 equivalent | 95.4% |
| | reaction: dichloromethane | Isobutyl boronic acid: 3.0 equivalents | |
| | (DCM) | TFA: 1.2 equivalents | |
| | | Dichloromethane (DCM): 50 mL | |
| | | Reaction temperature: 30° C. | |
| 7-3 | Solvent for condensation | Compound 10: 5.0 g, 1.0 equivalent | 93.2% |
| | reaction: acetonitrile | Isobutyl boronic acid: 3.0 equivalents | |
| | | TFA: 1.2 equivalents | |
| | | Acetonitrile: 50 mL | |
| | | Reaction temperature: 30° C. | |
| 7-4 | Solvent for condensation | Compound 10: 5.0 g, 1.0 equivalent | 81.6% |
| | reaction: acetone | Isobutyl boronic acid: 3.0 equivalents | |
| | | TFA: 1.2 equivalents | |
| | | Acetone: 50 mL, | |
| | | Reaction temperature: 30° C. | |
| 7-5 | Solvent: tetrahydrofuran | Compound 10: 5.0 g, 1.0 equivalent | 51.2% |
| | (THF) | Isobutyl boronic acid: 3.0 equivalents | |
| | | TFA: 1.2 equivalents | |
| | | Tetrahydrofuran (THF): 50 mL, | |
| | | Reaction temperature: 30° C. | |
| 7-6 | Condensation reaction at | Compound 10: 5.0 g, 1.0 equivalent | 93.89% |
| | 40° C. | Isobutyl boronic acid: 3.0 equivalents | |
| | | TFA: 1.2 equivalents | |
| | | Acetonitrile: 50 mL | |
| | | Reaction temperature: 40° C. | |
| 7-7 | Condensation reaction at | Compound 10: 5.0 g, 1.0 equivalent | 94.71% |
| | 50° C. | Isobutyl boronic acid: 3.0 equivalents | |
| | | TFA: 1.2 equivalents | |
| | | Acetonitrile: 50 mL | |
| | | Reaction temperature: 50° C. | |
| 7-8 | Dosage of trifluoroacetic acid | Compound 10: 5.0 g, 1.0 equivalent | 93.89% |
| | (TFA): 1.2 equivalents | Isobutyl boronic acid: 3.0 equivalents | |
| | | TFA: 1.2 equivalents | |
| | | Acetonitrile: 50 mL | |
| | | Reaction temperature: 40° C. | |
| 7-9 | Dosage of trifluoroacetic acid | Compound 10: 5.0 g, 1.0 equivalent | 90.5% |
| | (TFA): 0.6 equivalents | Isobutyl boronic acid: 3.0 equivalents | |
| | | TFA: 0.6 equivalent | |
| | | Acetonitrile: 50 mL | |
| | | Reaction temperature: 40° C. | |
| 7-10 | Dosage of acetonitrile: 10 | Compound 10: 5.0 g, 1.0 equivalent | 90.98% |
| | volumes | Isobutyl boronic acid: 3.0 equivalents | |
| | | TFA: 1.0 equivalent | |
| | | Acetonitrile: 50 mL | |
| | | Reaction temperature: 40° C. | |
| 7-11 | Dosage of acetonitrile: 3 | Compound 10: 5.0 g, 1.0 equivalent | 91.35% |
| | volumes | Isobutyl boronic acid: 3.0 equivalents | |
| | | TFA: 1.0 equivalent | |
| | | Acetonitrile: 15 mL, | |
| | | Reaction temperature: 40° C. | |
| 7-12 | Crystallization process: anti- | Reaction solution ACN/H₂O | Off-white solid |
| | solvent methyl tert-butyl ether | solution:MTBE = 1:10 (volume ratio) | Purity: 96.6% |
| | (MTBE) | Crystallization temperature: 0-10° C. | Yield: 79.0% |
| 7-13 | Crystallization process: anti- | Reaction solution ACN/H₂O | Off-white solid |
| | solvent isopropyl acetate | solution:isopropyl acetate = 1:10 | Purity: 97.2% |
| | | (volume ratio) | Yield: 55.8% |
| | | Crystallization temperature: 0-10° C. | |
| 7-14 | Crystallization process: anti- | Reaction solution ACN/H₂O | Gel |
| | solvent acetonitrile | solution:acetonitrile = 1:10 (volume | |
| | | ratio) | |
| | | Crystallization temperature: 0-10° C. | |

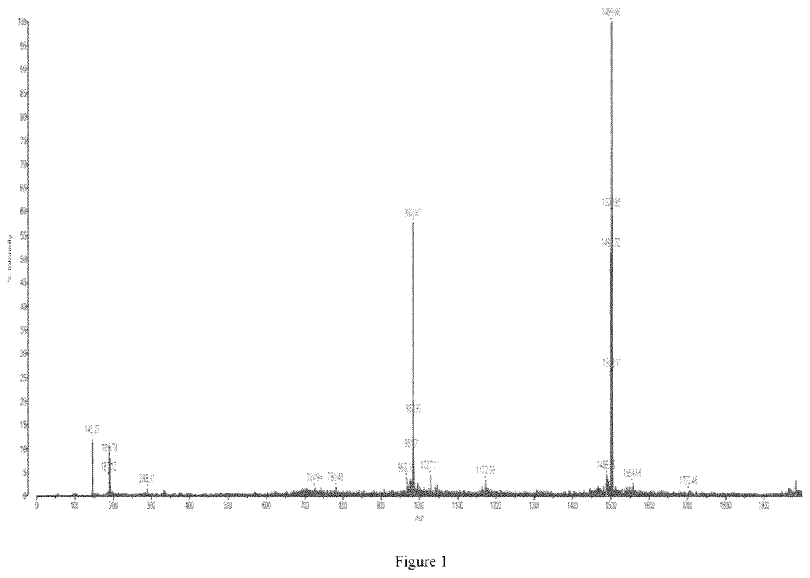

The elemental analysis results of the compound of Formula (I) were shown in Table 5, and the mass spectrum of the compound of Formula (I) was shown in FIG. 1.

TABLE 5

| | Theoretical | Measured | Result after | Absolute |
|---|---|---|---|---|
| | Value | Value | Adjusting for | Deviation |
| Element | (%) | (%) | Residue (%) | (%) |
| C | 57.61 | 57.09 | 57.16 | 0.45 |
| H | 6.65 | 6.63 | 6.64 | 0.01 |
| N | 11.20 | 11.32 | 11.33 | 0.13 |
| B | 2.16 | 2.18 | 2.18 | 0.02 |
| O | 22.38 | / | 22.69 | 0.31 |

| | |
|---|---|
| Note | The residue result is 0.13% |

Characterization data of the compound of formula (I):
¹H-NMR (400 MHZ, D20) δ=7.82-7.89 (0, 4H), 4.60 (d, J=8.5 Hz, 1H), 4.55 (dd, J=6.5, 2.0 Hz, 1H), 4.18 (s, 2H), 3.96-4.08 (m, 1H), 3.75-3.87 (m, 1H), 2.40-2.52 (m, 1H), 2.27-2.36 (m, 1H), 2.12-2.23 (0, 2H), 1.97-2.09 (0, 2H), 1.66-1.82 (m, 1H), 0.98-1.12 (m, 6H), 0.84-0.96 (m, 6H).
MS-negative ion: [M−H]⁻=1499.88

### Example 8 Activity Test of Compound (I) Against Recombinant Human Elastase (rhElastase)

Activation of protease: rhElastase was diluted to 2 μM with an activation buffer containing 1.25 μM Recombinant Mouse Active Cathepsin C Protein (mCathepsin C), and incubated at 37° C. for 2 h. After activation, the mature rhElastase was diluted to form a 3.33 nM rhElastase reaction solution. The compound was diluted in serial with DMSO and added to a 384-well plate (3-fold serial dilution, 10 concentration points, duplicate wells). For the 384-well plate containing the compound, columns 1-12 each was added with 15 μL of the reaction buffer and columns 13-24 each was added with 15 μL of the rhElastase reaction solution. The plate was pre-incubated at 25° C. for 30 min and added with 5 μL of the substrate to start the reaction. The final concentration of rhElastase is 2.5 nM, and the final concentration of the substrate is 3.125 μM. The final concentration of DMSO in the reaction system is 1%. After incubating at 37° C. for 60 min, the fluorescence value was read with an M4 microplate reader. The detection conditions are Ex/Em=380 nm/460 nm.

Result: The result shows that the IC₅₀ value of compound (I) against rhElastase is 1.318 nM.

### Example 9: Pharmacokinetic Experiment of Compound (I)

The appropriate amount of sodium citrate was weighed and dissolved in water to prepare a sodium citrate solution with a concentration of 1 mg/ml. After the addition of compound (I) (appropriate amount) under stirring, the sodium citrate solution was slowly added with 1 M sodium hydroxide solution to adjust the pH to 5.0±0.2. After stirring, dissolving and filtering, a stock solution of compound (I) with a concentration of 80 mg/ml was obtained, which is administrated by inhalation. The stock solution was diluted in a continuous gradient method to give a solution of compound (I) with a concentration of 0.4 mg/ml for intravenous administration.

21 male Sprague-Dawley rats was selected and divided into 7 groups (3animals/group). Group 1 was administered intravenously at a dose of 2 mg/kg, and groups 2-7 were administered by inhalation at a designed dose of 15 mg/kg (an actual delivered dose is 16.712 mg/kg). For animals in groups 1-2, blood samples were collected before administration and at 2 min, 5 min, 15 min, 30 min, 1 h, 2 h, 4 h, 6 h, 8 h, and 24 h after administration. For animals in groups 3-7, lung tissues and blood samples were collected at 0.25 h, 1 h, 2 h, 6 h, and 12 h after administration (i.e., 2.25 h, 3 h, 4 h, 8 h, and 14 h After the start of the drug administration). The LC-MS/MS was used to analyze the concentration of compound (I) in the plasma and lung tissues of rats, and the non-compartmental model (NCA) of the pharmacokinetic analysis software WinNonlin (8.0.0.3176) is used to analyze the drug concentrations in plasma and lung tissue and calculate the pharmacokinetic parameters.

The main pharmacokinetic parameters of compound (I) in Sprague-Dawley rats by intravenous/gavage/inhalation administration are shown in Table 6.

TABLE 6

| Administration | Dose | t_{1/2} | Tₘₐₓ | Cₘₐₓ | AUCₗₐₛₜ | |
|---|---|---|---|---|---|---|
| form | (mg/kg) | (h) | (h) | ng/mL | h*ng/mL | F % |
| Intravenous | 2 | 0.33 ± 0.10 | 0.03 ± 0.0 | 5331.49 ± 696.49 | 1107.18 ± 144.07 | / |
| Inhalation | 15 | 1.91 ± 1.05 | 2.03 ± 0.0 | 798.55 ± 333.20 | 1501.79 ± 486.138 | 18.09 |

After administered by inhalation to Sprague-Dawley rats, the exposure amounts of the compound (I) in the lungs and plasma of the rats are shown in Table 7.

TABLE 7

| Dose | | t_{1/2} | Tmax | | |
|---|---|---|---|---|---|
| (mg/kg) | Matrix | (h) | (h) | Cmax | AUClast |
| 15 | Lung | 2.69 | 2.25 | 67568.60 | 149415.60 |
| | Plasma | — | 2.25 | 320.58 | 622.35 |

It should be understood that the above examples are all exemplary and are not intended to cover all possible embodiments encompassed by the claims. Various modifications and changes can still be made on the basis of the above examples without departing from the scope of the present disclosure. Similarly, the various technical features of the above examples can also be arbitrarily combined to form additional examples that may not have been explicitly described herein. Therefore, the above examples only express several embodiments of the present invention and do not limit the protection scope of the present invention.

1. A process for preparing a compound of formula (I), comprising: 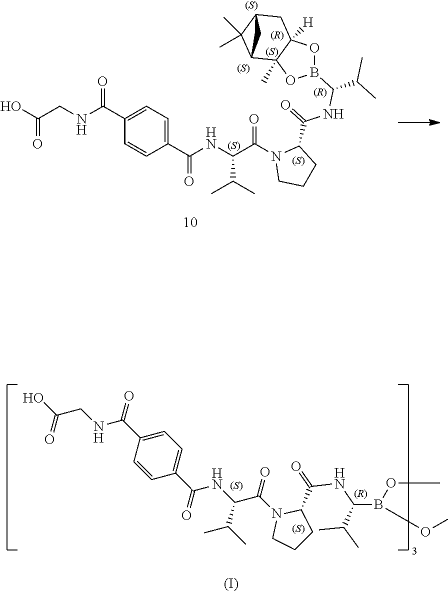
Step S7: reacting Compound 10 in the presence of a borylation reagent, an acidification reagent and a reaction solvent to obtain the compound of formula (I),
wherein the borylation reagent is selected from the group consisting of isobutyl boronic acid, phenylboronic acid and 4-trifluorophenylboronic acid;
the acidification reagent is selected from the group consisting of trifluoroacetic acid, hydrochloric acid, sulfuric acid and phosphoric acid; and
the reaction solvent is selected from the group consisting of acetonitrile, acetone, dichloromethane, 1,4-dioxane, methyl tert-butyl ether, alkane solvents (n-heptane, n-hexane, methylcyclohexane, petroleum ether), water, and combinations thereof.

2. The process according to claim 1, wherein step S7 further comprises a post-treatment comprising crystallization; preferably, a crystallization solvent is selected from the group consisting of 1,4-dioxane, tetrahydrofuran, 2-methyltetrahydrofuran, methyl tert-butyl ether, ethylene glycol dimethyl ether, dichloromethane, dimethyl sulfoxide, acetone, acetonitrile, water and combinations thereof; more preferably, the solvent used for the crystallization is selected from the group consisting of a mixture of methyl tert-butyl ether, acetonitrile and water, a mixture of acetonitrile and water and a mixture of acetone and water.

3. The process according to claim 1, further comprising: 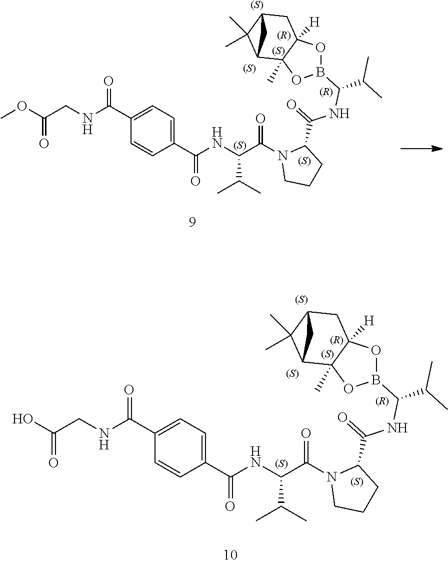
Step S6: reacting Compound 9 in the presence of a base to obtain Compound 10.

4. The process according to claim 3, wherein: the base is selected from the group consisting of sodium hydroxide, potassium hydroxide, lithium hydroxide and combinations thereof, preferably the base is lithium hydroxide;
preferably, step S6 further comprises a post-treatment step comprising an extraction;
preferably, the extraction includes the addition of water and an extractant into the reaction solution for extracting after the reaction is completed;
preferably, the extractant is selected from the group consisting of ethyl acetate, cyclopentyl methyl ether, isopropyl acetate, methyl tert-butyl ether, methyl acetate, propyl acetate and combinations thereof; more preferably, the extractant is methyl tert-butyl ether;
preferably, the post-treatment step of step S6 comprises crystallization;
preferably, a crystallization solvent is selected from the group consisting of 1,4-dioxane, n-heptane, tetrahydrofuran, ethyl acetate, isopropyl acetate, 2-methyltetrahydrofuran, methyl tert-butyl ether, ethylene glycol dimethyl ether, dichloromethane, dimethyl sulfoxide, acetonitrile and combinations thereof; more preferably, the crystallization solvent is a mixture of n-heptane and tetrahydrofuran.

5. The process according to claim 3, further comprising: 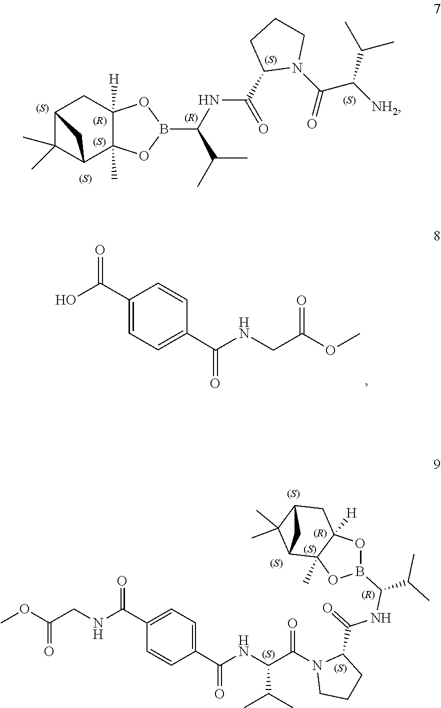
Step S5: reacting Compound 7 or the salt thereof with compound 8 in the presence of a first condensation reagent to obtain compound 9.

6. The process according to claim 5, wherein the first condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof;
preferably, the condensation reagent is T₃P or T₄P;
preferably, the condensation reagent is T₄P.

7. The process according to claim 5, further comprising: 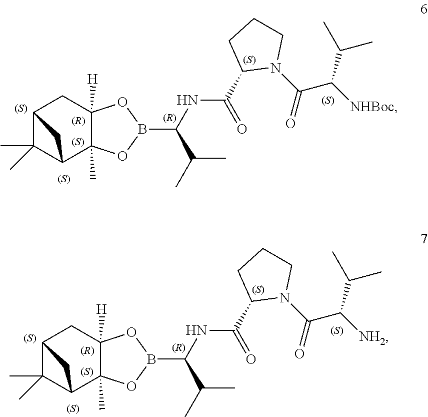
Step S4: reacting Compound 6 in the presence of an acid to obtain a salt of compound 7.

8. The process according to claim 7, wherein the acid is selected from the group consisting of hydrochloric acid, trifluoroacetic acid (TFA), sulfuric acid, p-toluenesulfonic acid, formic acid and combinations thereof; preferably, the acid is hydrochloric acid.

9. The process according to claim 7, further comprising: 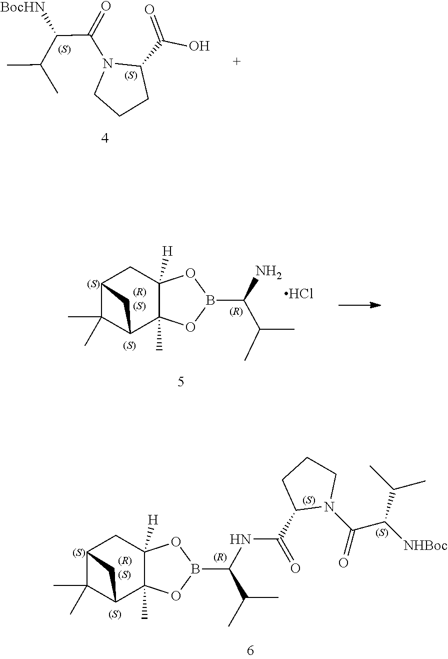
Step S3: reacting Compound 4 with compound 5 in the presence of a second condensation reagent to obtain compound 6.

10. The process according to claim 9, wherein the second condensation reagent is selected from the group consisting of DIC, DCC, EDCI, HATU, HBTU, HCTU, TBTU, BOP, PyBOP, DPP-Cl, DPPA, BOP-Cl, T₃P, T₄P, CDI and combinations thereof;
preferably, the condensation reagent is T₃P or T₄P;
more preferably, the condensation reagent is T₄P.
